(19) United States
(12) Patent Application Publication
(10) Pub. No.: US 2008/0154061 A1
(43) Pub. Date: Jun. 26, 2008

(54) Method For Producing a Xylylenediamine

(75) Inventors: Martin Ernst, Heidelberg (DE); Kirsten Wenz, Mannheim (DE); Randolf Hugo, Dirmstein (DE); Johann-Peter Melder, Bohl-Iggelheim (DE); Thilo Hahn, Freimersheim (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 11/816,878

(22) PCT Filed: Feb. 23, 2006
(86) PCT No.: PCT/EP06/60226

§ 371 Date: Aug. 22, 2007

(30) Foreign Application Priority Data
Feb. 24, 2005 (DE) 10 2005 008929.1

(51) Int. Cl. C07C 211/07

(52) U.S. Cl. 564/372

(57) ABSTRACT

A process for preparing a xylylenediamine by heterogeneously catalyzed hydrogenation of a phthalonitrile, wherein the hydrogenation is carried out in the presence of a nickel catalyst, of water, of an alkali metal hydroxide and of an ether as a solvent, at an absolute pressure in the range from 1 to 100 bar, at a temperature in the range from 40 to 150° C. and without addition of ammonia.

The present invention relates to a process for preparing a xylylenediamine by heterogeneously catalyzed hydrogenation of a phthalonitrile.

Xylylenediamine(bis(aminomethyl)benzene) is a useful starting material, for example, for the synthesis of polyamides, epoxy hardeners, or as an intermediate for preparing isocyanates.

The term “xylylenediamine” (XDA) comprises the three isomers ortho-xylylenediamine, meta-xylylenediamine (MXDA) and para-xylylenediamine.

The term “phthalonitrile” (PN) comprises the three isomers 1,2-dicyanobenzene=o-phthalonitrile, 1,3-dicyanobenzene=isophthalonitrile=IPN and 1,4-dicyanobenzene=terephthalonitrile.

The phthalonitriles are solids (for example, isophthalonitrile (IPN) melts at 161° C.) and have relatively poor solubilities in many organic solvents.

The two-stage synthesis of xylylenediamine by ammoxidation of xylene and subsequent hydrogenation of the resulting phthalonitrile is known in principle.

U.S. Pat. No. 4,482,741 (UOP Inc.) describes the hydrogenation of PN in the presence of ammonia, a supported Co/Ti catalyst and XDA as a solvent.

On page 6, last paragraph, DE-A-21 64 169 (Mitsubishi Gas Chemical Co., Inc.) describes the hydrogenation of IPN to meta-XDA in the presence of an Ni catalyst and/or Co catalyst in ammonia as a solvent.

JP-B-46008283 (Toray Industries Inc.; ACS Abstract 75:5222) relates to the hydrogenation of nitriles such as aminocapronitrile to primary amines in the presence of lead-containing nickel or cobalt catalysts.

U.S. Pat. No. B1-6,660,887 (Solutia Inc.) describes the preparation of 3-dimethylaminopropyl-amine (DMAPA) from N,N-dimethylaminopropionitrile (DMAPN) at low pressure in the presence of a nickel catalyst.

FR-A1-2 722 784 (Rhone Poulenc) teaches in particular the hydrogenation of dinitriles such as adiponitrile to diamines in the presence of Ti-doped Raney nickel catalysts.

U.S. Pat. No. 3,862,911 (and DE-A-2 260 978) (Rhone Poulenc) describes Ni/Cr/Fe/Al catalysts for hydrogenating nitriles, in particular adiponitrile. In example 6B, the hydrogenation of IPN to MXDA in ethanol succeeds at 85° C. and 40 bar with a yield of only 75%.

ACS Abstract No. 139:381881 (JP-A2-2003 327563) (Mitsubishi Gas) discloses a process for continuously hydrogenating aromatic dinitriles in an ammonia-containing solvent such as m-xylol in a “fixed bed irrigation liquid type reactor” and in the presence of nickel or cobalt catalysts.

EP-A1-1 449 825 (Mitsubishi Gas Chem. Comp.) describes a two-stage preparation of aromatic diamines from aromatic dinitriles such as IPN in the presence of a Pd catalyst in the first stage and in the presence of an Ni or Co catalyst in the second stage.

U.S. Pat. No. 2,970,170 and GB-B-821 404 (California Research Corp.) relate to a multistage production process for xylylenediamines starting from the corresponding phthalic acids. For the dinitrile hydrogenation, for example in the presence of cobalt or nickel catalysts, pressures in the range from 1500 to 10 000 psig (103.4-689.5 bar), particularly from 2000 to 5000 psig (137.9-344.7 bar), and temperatures in the range from 180 to 400° F. (82 to 204° C.) are taught (U.S. Pat., column 3, lines 65-71).

EP-A1-1 454 895 (Mitsubishi Gas Chem. Comp.) describes a two-stage process for hydrogenating dicyanobenzenes at pressures of from 5 to 300 bar, in particular from 10 to 200 bar, in the presence of supported or unsupported Co, Ni, Pd, Ru or Rh catalysts, preferably in the presence of ammonia and optionally in the presence of additives such as alkali metal hydroxides or alkaline earth metal hydroxides.

U.S. Pat. No. B1-6,476,267 (Sagami Chemical Research Center) relates to the preparation of aromatic primary amines from nitriles such as IPN in the presence of supported Ni catalysts and polar solvents, preferably in the presence of NH₃, at pressures of from 0.1 to 50 kg/cm² G (from 0.1 to 49 bar, for example ≦19 kg/cm²G (18.6 bar) and temperatures up to 200° C.

Support material of the Ni catalyst is silica, alumina or activated carbon, preferably silica (all examples).

According to example 22, the hydrogenation of IPDN succeeds in methanol in the presence of NH₃ over a silica-supported Ni catalyst at 170° C. and 15 kg/cm²G (14.7 bar) in 79.5% yield.

GB-B-810 530 (P. B. Brindley et al.) teaches the hydrogenation of iso- or terephthalonitrile in the presence of ammonia, nickel or cobalt catalysts and aromatic hydrocarbons, water, DMF, methanol or ethanol as a solvent. The pressure is up to 200 atm. (203 bar).

EP-A1-913 388 (Air Products) relates to the hydrogenation of nitrites such as DMAPN to amines in the presence of Raney cobalt catalysts, LiOH and water, and in the absence of organic solvents, at pressures in the range from 1 to 300 bar, in particular from 5 to 80 bar.

Disadvantages arise here as a result of the complexity of feeding the reactant nitrile, in the case that it is a solid, to the reactor, and as a result of the reactant nitrile and/or intermediates such as imines forming undesired by-products with the product amine to too high a degree.

ACS Abstract No. 91:91334 (JP-A2-54 041 804) (Takeda Chem. Ind.) relates to the hydrogenation of nitrites such as IPN in solvent mixtures composed of alcohols and cyclic hydrocarbons over Raney Co catalysts or Raney Ni catalysts at, for example, 105-115 kg/cm² (103-113 bar).

U.S. Pat. No. 3,647,054 (ACS Abstract No. 73:130762) (Japan Gas-Chemical Comp.) describes the hydrogenation of phthalonitrile in liquid ammonia and in the presence of methanol over Raney Ni at 200 atm. (203 bar) and the subsequent purification of the crude product.

ACS Abstract No. 74:31537 (JP B4-45 03 0088) (Toray Ind.) teaches the hydrogenation of aminocapronitrile in liquid ammonia over a modified Raney Co catalyst.

ACS Abstract No. 59:61849 (JP 38 00 8719) (Toho Rayon Co.) relates to the hydrogenation of terephthalonitrile and isophthalonitrile in methanol over Raney Ni at 30° C. in the presence of KOH.

U.S. Pat. No. 3,544,485 (Toyo Rayon) and ACS Abstract No. 73:109473 (JP-B445 016 098) (Toray Ind.) describe methods for activating Raney alloys.

DE-A1-100 65 031 (Degussa AG) relates to the use of Raney catalysts in the form of hollow bodies in hydrogenation processes.

The six German patent applications having the reference numbers 10341615.3, 10341632.3, 10341614.5, 10341633.1, 10341612.9 and 10341613.7 (BASF AG) of Sep. 10, 2003, and the two German patent applications having the reference numbers 102004042947.2 and 102004042954.5 (BASF AG) of Sep. 2, 2004 likewise relate to processes for preparing XDA.

The German patent application having the reference number 102005003315.6 (BASF AG) of 01.24.05 describes a process for preparing a xylylenediamine by heterogeneously catalyzed hydrogenation of a phthalonitrile in the presence of a cobalt skeletal catalyst.

It is an object of the present invention to discover an improved, economically viable process for preparing a xylylenediamine. The process should overcome one or more disadvantages of the prior art processes. The xylylenediamine, especially MXDA, should be obtained in high yield, especially space-time yield, selectivity, purity and/or color quality.

[Space-time yields are reported in “amount of product/(volume of catalyst·time)” (kg/(I_{cat}·h)) and/or “amount of product/(reactor volume·time)” (kg/(I_{reactor}·h)].

Accordingly, a process has been found for preparing a xylylenediamine by heterogeneously catalyzed hydrogenation of a phthalonitrile, which comprises carrying out the hydrogenation in the presence of a nickel skeletal catalyst, of water, of an alkali metal hydroxide and of an ether as a solvent, at an absolute pressure in the range from 1 to 100 bar, at a temperature in the range from 40 to 150° C. and without addition of ammonia.

The process according to the invention preferably finds use for preparing meta-xylylenediamine (MXDA) by hydrogenating isophthalonitrile (IPN).

Advantages of the process according to the invention include the lower level of apparatus and safety expense and complexity resulting from the method without NH₃ addition and the low-pressure method, and thus lower fixed costs (investment) and variable costs.

In addition, in the selective process according to the invention, particularly small amounts of by-products, for example products having a higher boiling point than xylylenediamine (at the same pressure) and amidines, for example of the formula I, and their subsequent products (dimers of MXDA of the formula II).

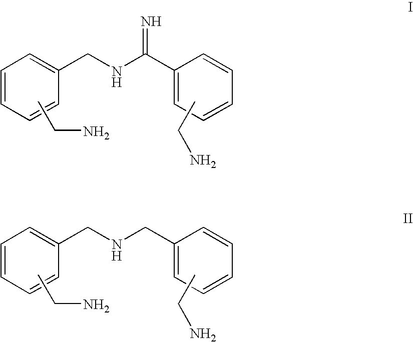

The PN used as a reactant in the process may be synthesized in a preceding stage by ammoxidation of the corresponding xylene isomer. Such synthetic processes are described, for example, in the BASF patent applications EP-A-767 165, EP-A-699 476, EP-A-222 249, DE-A-35 40 517 and DE-A-37 00 710, and in the abovementioned eight BASF patent applications for preparing XDA of Sep. 10, 2003 and Sep. 2, 2004.

The process according to the invention can be performed as follows:

The PN feedstock is used preferably in a purity of ≧90% by weight, in particular ≧98% by weight, for example from 98.2 to 99.9% by weight. Such purities may be achieved, for example, by distillation or rectification of commercially available material.

The hydrogenation process according to the invention is preferably carried out in the presence of from 0.5 to 15% by weight, particularly from 2 to 10% by weight, very particularly from 2.5 to 7% by weight, in particular from 3 to 5% by weight, of water, based in each case on the PN used.

For the hydrogenation of the phthalonitrile to the corresponding xylylenediamine (o-, m- or p-xylylenediamine) according to the equation

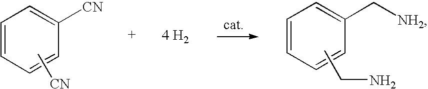

the PN is dissolved and/or suspended in an ether. To increase the rate of dissolution and/or to increase the amount of dissolved PN, the dissolution operation may be effected at elevated temperature, for example from 50 to 145° C.

In the process according to the invention, preference is given to using from 15 to 75% by weight, in particular from 20 to 50% by weight, of solutions and/or suspensions of PN in the solvent or solvent mixture.

The solvents and/or suspension media used are preferably a C₄₋₂-dialkyl ether and/or C₃₋₁₂-alicyclic ether, in particular a C₄₋₆-dialkyl ether and/or C₄₋₆-alicyclic ether.

Examples thereof are methyl tert-butyl ether (MTBE), diethyl ether (DEE), di-n-propyl ether, di-n-butyl ether, 1,2-dimethoxyethane, 1,2-diethoxyethane, tetrahydrofuran (THF), 2-methyl-THF, tetrahydropyran, 1,3-dioxepane, 1,4-dioxane, 1,3-dioxane and 1,3-dioxolane. Particular preference is given to THF.

The solvents and/or suspension media used may also be a mixture of two or more of the solvents mentioned.

According to the invention, the catalyst used for the hydrogenation is a nickel skeletal catalyst.

Typical examples of such catalysts are Raney™ nickel catalysts. In this case, the active catalyst is prepared as “metal sponge” from a binary alloy of nickel and, if appropriate, further elements, with, for example aluminum by leaching out a partner with acid or alkali. Residues of the original alloy partner often have a synergistic effect.

The catalysts used in the process according to the invention are preferably prepared starting from an alloy of nickel and a further alloy component which is soluble in alkalis. For this soluble alloy component, preference is given to using aluminum, but it is also possible to use other components such as zinc and silicon or mixtures of such components.

To activate the catalysts, the soluble alloy component is extracted fully or partly with alkali, for which, for example, aqueous sodium hydroxide solution may be used. The catalyst may then be washed, for example, with water or organic solvent.

In the catalyst, one or more further elements may be present as promoters. Examples of promoters are metals of transition groups IB, VIB and/or VIII of the Periodic Table, such as chromium, iron, molybdenum, cobalt, copper, etc.

The catalysts may be activated by leaching out the soluble component (typically aluminum) either in the reactor itself or before charging into the reactor. The preactivated catalysts are air-sensitive and pyrophoric and are therefore generally stored and handled under a medium, for example water, an organic solvent or a substance which is present in the inventive reaction (solvent, reactant, product), or embedded into an organic compound which is solid at room temperature.

The catalysts may be used in the form of powder for suspension hydrogenations, in the form of granules or in the form of moldings such as tablets or extrudates for fixed bed reactors.

According to the invention, preference is given to using a nickel skeletal catalyst which has been obtained from an Ni/Al alloy by leaching with aqueous alkali metal hydroxide solution, for example sodium hydroxide solution, and subsequent washing with water, and preferably comprises at least one of the elements Fe, Cr as promoters.

In addition to nickel, such activated catalysts typically also comprise

1-30% by weight of Al, particularly 2-20% by weight of Al, very particularly 5-14% by weight of Al, and
0-10% by weight of Cr, particularly 0.1-7% by weight of Cr, very particularly 1-4% by weight of Cr, and/or
0-10% by weight of Fe, particularly 0.1-7% by weight of Fe, very particularly 1-4% by weight of Fe,
the weight data each being based on the total catalyst weight.

The catalyst used in the process according to the invention may, for example, be an A 4000 nickel skeletal catalyst from Johnson Matthey.

This catalyst has the following composition:

Al: ≦4% by weight, Ni: ≧80% by weight, Fe: 1-4% by weight, Cr: 1-4% by weight.

The nickel skeletal catalyst used preferably comprises no lead (Pb) and/or no cobalt (Co) and/or no metal of transition group IVB, i.e. no Ti, Zr and/or Hf.

The PN is converted in the presence of alkali metal hydroxide (MOH), in particular from 0.001 to 5 mol % of MOH, very particularly from 0.002 to 1.5 mol % of MOH, more preferably from 0.005 to 1.2 mol % of MOH, for example 1 mol % of MOH, based in each case on the PN used.

In a preferred embodiment, the appropriate amount of MOH is used in the form of an aqueous solution, for example in the form of at from 1 to 25% by weight aqueous solution.

Possible alkali metals M are Li, Na, K, Rb and Cs. Preferably, M═K or Na. More preferably, M═K.

It is also possible to use mixtures of two or more of the alkali metal hydroxides mentioned (MOH), in which case the abovementioned MOH amounts are then based on the sum of the alkali metal hydroxides. For example, an NaOH—KOH mixture may be used.

In a particular embodiment, the catalyst used is treated beforehand with alkali metal hydroxide (M′OH) or a mixture of two or more alkali metal hydroxides M′OH, for example a mixture of NaOH and KOH. This treatment is particularly advantageous when the hydrogenation is carried out in the absence of MOH in the initially charged reaction mixture.

This treatment of the catalyst with M′OH may be effected by processes known to those skilled in the art, for example by saturating the catalyst with M′OH, for example from 0.01 to 5.0% by weight of M′OH (based on the support material), in the presence of a suitable solvent, for example water (EP-A1-913 388, U.S. Pat. No. 6,429,338, U.S. Pat. No. 3,636,108).

Possible alkali metals M′ are Li, Na, K, Rb and Cs. Preferably, M′═K or Na. More preferably, M′═K.

The hydrogenation is carried out without addition of ammonia.

The reaction temperature of the hydrogenation is in the range from 40 to 150° C., preferably from 50 to 120° C., in particular from 60 to 110° C., very particularly from 70 to 105° C., for example from 80 to 100° C.

The absolute pressure in the hydrogenation is in the range from 1 to 100 bar, preferably from 2 to 80 bar, in particular from 5 to 60 bar, very particularly from 10 to 50 bar, for example from 20 to 40 bar.

The hydrogenation is preferably carried out in one reaction stage. In other words, a plurality of hydrogenation stages, as taught, for example, in EP-A1-1 449 825 and EP-A1-1 454 895, need not be employed.

The reactors used for the process according to the invention may, for example, be customary high-pressure autoclaves.

For the hydrogenation, the reactors (for example fixed bed or suspension method) and processes (continuous, semicontinuous (semibatchwise), discontinuous (batchwise)) which are known to those skilled in the art for this reaction may be employed. In the suspension method, preference is given to a continuous process or semibatchwise process.

In the fixed catalyst bed method, both the liquid phase and the trickle method are possible. Preference is given to a trickle method.

The hydrogenation reactor may be operated in straight pass. Alternatively, a circulation method, in which a portion of the reactor effluent is recycled to the reactor inlet, is also possible, preferably without preceding workup of the circulation stream. This allows optimal dilution of the reaction solution to be achieved, which has a favorable effect on the selectivity. In particular, the circulation stream may be cooled in a simple and inexpensive manner by means of an external heat transfer and the heat of reaction may thus be removed. The reactor can also be operated adiabatically, in which case the temperature rise of the reaction solution can be restricted by the cooled circulation stream. Since the reactor itself then does not have to be cooled, a simple and inexpensive design is possible. An alternative is a cooled tube bundle reactor.

In the preferred suspension method in a semibatchwise process, preference is given to initially charging the nickel skeletal catalyst, the alkali metal hydroxide and water in the reactor and subsequently feeding the phthalonitrile in the solvent under the reaction conditions established (pressure, temperature) over a certain period (for example 2-8 h) (semicontinuous method).

In a particular embodiment, especially of this method, the XDA corresponding to the PN used is additionally initially charged, for example in amounts of from 500-1500% by weight based on PN used.

The XDA corresponding to the PN used is ortho-XDA in the case of the ortho-dinitrile, MXDA in the case of the meta-dinitrile and para-XDA in the case of the para-dinitrile.

The conversions of PN achievable with the process according to the invention are in the range of ≧95%, in particular ≧99%, for example from ≧96 to 99.9% or from 99.5 to 100%, at selectivities (for the formation of XDA) in the range of ≧80%, in particular ≧85%, for example from 86 to 99.5% or from 90 to 99%.

The reaction effluent freed of the solvent comprises in particular ≦2% by weight, very particularly ≦1% by weight, for example from 0 to 0.5% by weight, of amidines of the formula I and/or products having a higher boiling point than XDA, for example the corresponding (bisaminodialkyl)diarylamine II.

After the process according to the invention has been carried out, the XDA may be isolated, for example, by distillation or rectification.

### EXAMPLES

### Example 1

In a 300 ml high-pressure autoclave with magnetic sparging stirrer, sampling neck, temperature control and an inlet for the continuous feeding of reactants, 60 g of THF, 1.19 g of water-moist undoped Raney™ nickel and 0.021 g of KOH were combined in 1.73 g of water.

The autoclave was closed, the mixture was inertized and hydrogen was injected to 10 bar. The mixture was heated to 10° C. under autogenous pressure and with stirring (500 rpm). When this temperature was attained, hydrogen was injected to 36 bar and the stirrer rotation rate increased to 1200 rpm. Subsequently, a solution of 7.2 g of IPN in 83 g of THF was pumped in over 5 h, and hydrogen was fed in continuously (while maintaining the pressure at 32-36 bar).

After 5 h, a sample was taken. GC analysis of the samples gave a conversion of 100% and a selectivity of 96.5%. No formation of high boilers was observed. The mixture was kept under the same conditions for a further 5 h after the end of the metered addition without the selectivity falling.

### Example 2

### Simulation of the Backmixed Method

In a 300 ml high-pressure autoclave with magnetic sparging stirrer, sampling neck, temperature control an an inlet for the continuous feeding of reactants, 60 g of MXDA, 5.95 g of Raney™ nickel A4000 from Johnson Matthey, which has been washed beforehand with MXDA, to free it of water, and 0.1 g of KOH were combined in 0.5 g of water.

The autoclave was closed, the mixture was inertized and hydrogen was injected to 10 bar. The mixture was heated to 10° C. under autogenous pressure and with stirring (500 rpm). When this temperature was attained, hydrogen was injected to 36 bar and the stirrer rotation rate increased to 1200 rpm. Subsequently, a solution of 7.2 g of IPN in 83 g of THF was pumped in over 1 h, and hydrogen was fed in continuously (while maintaining the pressure at 32-36 bar).

After 1 h, a sample was taken. GC analysis of the samples gave an MXDA content of 99.1% at a conversion of 100%. The highboiler content was 0.24% (in GC area %). The mixture was kept under the same conditions for a further 2 h after the end of the metered addition without the selectivity falling.

1. A process for preparing a xylylenediamine by heterogeneously catalyzed hydrogenation of a phthalonitrile, which comprises
carrying out the hydrogenation in the presence of a nickel skeletal catalyst, of water, of an alkali metal hydroxide and of an ether as a solvent, at an absolute pressure in the range from 1 to 100 bar, at a temperature in the range from 40 to 150° C. and without addition of ammonia.

2. The process according to claim 1 for preparing meta-xylylenediamine (MXDA) by hydrogenating isophthalonitrile (IPN).

3. The process according to claim 1, wherein the hydrogenation is carried out in one reaction stage.

4. The process according to claim 1, wherein the hydrogenation is carried out at an absolute pressure in the range from 5 to 60 bar.

5. The process according to claim 1, wherein the hydrogenation is carried out at a temperature in the range from 60 to 120° C.

6. The process according to claim 1, wherein the nickel skeletal catalyst has been obtained from an Ni/Al alloy by leaching with aqueous alkali metal hydroxide solution and washing.

7. The process according to claim 1, wherein the nickel skeletal catalyst comprises Fe and/or Cr as a promoter.

8. The process according to claim 1, wherein the nickel skeletal catalyst, in addition to nickel, further comprises 1-30% by weight of Al, 0.1-10% by weight of Cr and/or 0.1-10% by weight of Fe, based on the total catalyst weight.

9. The process according to claim 1, wherein the hydrogenation is carried out in the presence of a C₄₋₁₂-dialkyl ether and/or C₃₋₁₂-alicyclic ether as the solvent.

10. The process according to claim 1, wherein the hydrogenation is carried out in the presence of tetrahydrofuran (THF) as the solvent.

11. The process according to claim 1, wherein the hydrogenation is carried out in the presence of from 0.001 to 5 mol % of alkali metal hydroxide based on the phthalonitrile used.

12. The process according to claim 1, wherein the alkali metal hydroxide is used in the form of an aqueous solution.

13. The process according to claim 1, wherein the alkali metal hydroxide used is potassium hydroxide (KOH) or sodium hydroxide (NaOH).

14. The process according to claim 1, wherein the alkali metal hydroxide used is a mixture of potassium hydroxide (KOH) and sodium hydroxide (NAOH).

15. The process according to claim 1, wherein the heterogeneous catalyst used has been treated beforehand with an alkali metal hydroxide or a mixture of alkali metal hydroxides.

16. The process according to claim 1, wherein the heterogeneous catalyst used has been treated beforehand with potassium hydroxide (KOH).

17. The process according to claim 1, wherein a semibatchwise method and not a batchwise method is carried out.

18. The process according to claim 2, wherein a continuous method and not a semibatchwise or batchwise method is carried out.

19. The process according to claim 1, wherein the hydrogenation is carried out in the presence of added xylylenediamine which corresponds to the phthalonitrile used.

20. The process according to claim 1, wherein the hydrogenation is carried out in the presence of from 0.5 to 15% by weight of water based on the phthalonitrile used.
